(19) United States
(12) Patent Application Publication
(10) Pub. No.: US 2009/0324743 A1
(43) Pub. Date: Dec. 31, 2009

(54) PULMONARY DRUG DELIVERY

(75) Inventors: Katharine Christine Carter, Edinburgh (GB); Alexander Balfour Mullen, East Kilbride (GB); Valerie Anne Ferro, Lanarkshire (GB)

Correspondence Address: INTELLECTUAL PROPERTY / TECHNOLOGY LAW, PO BOX 14329, RESEARCH TRIANGLE PARK, NC, 27709, US

(73) Assignee: UNIVERSITY OF STRATHCLYDE

(21) Appl. No.: 12/492,063

(22) Filed: Jun. 25, 2009

Related U.S. Application Data
(63) Continuation-in-part of application No. PCT/GB09/00539, filed on Feb. 26, 2009.
(60) Provisional application No. 61/076,494, filed on Jun. 27, 2008.

(51) Int. Cl. A61K 33/24

(52) U.S. Cl. 424/649

(57) ABSTRACT

The present invention relates to formulations for delivery of drugs to the respiratory tract, especially the lungs, as well as improved methods of treating subjects suffering or predisposed to suffering from lung conditions/diseases. The invention is particularly directed to lung cancer treatment using, for example, cytotoxic platinum-based drugs.

### CROSS-REFERENCE TO RELATED APPLICATIONS

This application is a continuation-in-part of International Patent Application No. PCT/GB09/000,539 filed Feb. 26, 2009, and also claims priority to U.S. Provisional Patent Application No. 61/076,494 filed Jun. 27, 2008. The disclosures of all of the foregoing applications are hereby incorporated herein in their respective entireties, for all purposes, and the priority of all such applications is hereby claimed under the provisions of 35 USC §120.

### FIELD OF THE INVENTION

The present invention relates to formulations for delivery of drugs to the respiratory tract, especially the lungs, as well as improved methods of treating subjects suffering or predisposed to suffering from lung conditions/diseases. The invention is particularly directed to lung cancer treatment using, for example, cytotoxic platinum based drugs.

### BACKGROUND TO THE INVENTION

The lung is an ideal target for delivery of therapeutics such as drugs, peptides, proteins, carbohydrates, nucleic acid or other entities, as it has a large surface area for absorption (approximately 100 m² humans, a relatively thin epithelium (50-60 μm in the upper airways) and a rich blood supply (Cryan et al. 2007). Both active and passive transport mechanisms occur in lung epithelial cells and there is a large population of macrophages, which can be used to facilitate uptake of therapeutics if they are incorporated into vesicular drug delivery systems.

One particularly problematic area of treatment is in terms of treating cancers using cytotoxic drugs, such as the platinum based drugs including cisplatin. Systemic administration of cytotoxic drugs is less than desirable, as this can lead to death of cells other than the cancer cells to be treated. Thus, there is the desire to administer such cytotoxic drugs directly to the tumour to be treated. In terms of lung cancer, there is the possibility of delivering the drug directly to the lung, in order to minimise sustained distribution of the drug. However, formulations need to be developed which would allow such a delivery.

Various patents and patent applications (PCT WO2007/099377, PCT WO 2004/054499, and U.S. Pat. No. 6,511,676) describe formulations based on the use of liposomes to deliver platinum based drugs such as cisplatin. However, liposomal formulations are expensive to produce, less stable as phospholipids can be chemically unstable, and manufacturing methods are more complicated. Thus there is thus a desire to develop alternative formulations.

The present inventors previously described the use of non-ionic surfactant vesicles to deliver drugs by intravenous administration WO96/04890. However, there was no suggestion that such vesicles could be delivered by an inhalation route, or teaching that the vesicles could be used to entrap cytotoxic drugs useful for treating, for example, lung cancer.

### SUMMARY OF THE INVENTION

The present invention is based on work by the present inventors which shows that formulations comprising drug loaded non-ionic surfactant vesicles are particularly useful in delivering a drug by inhalation means. The inventors have shown this to be particularly efficacious for delivering antibiotics and cytotoxic drugs, such as cisplatin to the lung.

Thus, in a first aspect there is provided a formulation for pulmonary delivery comprising active agent loaded non-ionic surfactant vesicles for delivery to the respiratory system.

Typically the composition is intended for treating or preventing a respiratory infection or disease, such as a cancer, viral, fungal or bacterial infection, immune disease, inflammatory disease, allergy or the like. However, the formulations may also find application in diagnosis.

There is also provided use of a formulation for pulmonary drug delivery comprising active agent loaded non-ionic surfactant vesicles for delivering to the respiratory system for preventing, diagnosing or treating a respiratory condition or disease.

In a further aspect there is provided a method of treating a disease or condition of the respiratory tract, comprising administering to a region of the respiratory system, such as the lung, an effective amount of active agent loaded non-ionic surfactant vesicles.

In principal, the vesicles may be formed in any manner known in the art and appropriate to the active agent to be delivered. For example, vesicles can be formed using either a “homogenisation” method or a “freeze-dried” method, both methods being known in the art. In the homogenisation method a required quantity of non-ionic surfactant material in a desired molar ratio can be processed in one of the following ways: Dry powders (i.e. non-ionic surfactant material) are hydrated with a solution of the active agent for entrapment at a desired temperature, in the range from 0 up to 150° C. and homogenised at the required speed and for the required length of time to produce the desired vesicle characteristics. Alternatively the lipid material can be melted by the application of heat (e.g. temperature range 40° C.-150° C.) prior to hydration with the required solution at the necessary temperature. The suspension can then be homogenised at the required speed and for the required length of time to produce vesicles having desired characteristics.

In the freeze-dried method, a freeze dried preparation can be made in one of the following ways:

The required quantity of vesicle constitutents in a desired molar ratio can be dissolved in an appropriate organic solvent (e.g. t-butyl alcohol) prior to filtration, for example, through a porous membrane (e.g. 0.2 μm). The surfactant solution can then be frozen and freeze-dried for the time required for complete removal of organic solvent. Resultant lyophilised product can then be hydrated with a solution of the active agent to be entrapped and shaken at the required temperature to produce a vesicle suspension. Alternatively, vesicle suspensions are produced by the homogenisation process described above, filtered through a porous membrane (e.g. 0.2 μm), and then lyophilised to remove the aqueous solvent. The resultant lyophilised product can then be hydrated with the required solution and shaken at the required temperature to produce a vesicle suspension.

The vesicle is preferably formed with a sterol such as cholesterol or ergosterol, together with a non-ionic surfactant. It is generally also necessary to include a charged species such as a fatty acid within the vesicle formulation in order to prevent clumping of the vesicles. Suitable charges species include dicetylphosphate, stearic acid and palmitic acid.

It has been found particularly advantageous to employ a non-ionic surfactant. This may be a mono, di-, tri- or poly (up to 10) glycerol mono- or di-fatty acid ester (e.g. C₁₀-C₂₀ fatty acid ester) such as triglycerol monostearate; or may be a polyoxyethylene ether preferably comprising from 1 to 10 oxyethylene moieties with a C₁₀-C₂₀ normal or branched alkyl chain such as to provide a hydrophilic head portion and a hydrophobic tail portion.

Preferred examples are: triglycerol monostearate, hexaglycerol distearate, diethylene glycol mono n-hexadecylether, tetraethylene glycol mono n-hexadecylether, hexaethylene glycol mono n-hexadecylether, decaethylene glycol mono n-hexadecylether; cholesterol, and an amphiphile such as dicetyl phosphate or a fatty acid.

It is now considered that vesicle formulations comprising a non-ionic surfactant, cholesterol and amphiphile e.g. dicetyl phosphate or a fatty acid can be present in a molar ratio of 3-5:1-4:0-4 respectively.

Preferred vesicle formulations comprise a non-ionic surfactant, cholesterol and dicetyl phosphate or a fatty acid selected from stearic or palmitic acid and these are advantageously present in a molar ratio of 3-5:2-4:0-3 respectively.

One preferred formulation comprises 600-2000 μmol lipid non-ionic vesicle suspension using a 3:3:1 molar ratio of hexaethylene glycol mono n-hexadecylether: cholesterol: dicetyl phosphate.

The vesicle diameter determined as described herein has now been found to be in the range of from 100 to 2500 nm and may therefore be considerably larger than 1000 nm. Preferably the vesicle diameter lies in the range of from 100 to 1000 nm and more preferably from 200 to 600 nm.

Hydrophilic active agents will generally be soluble in the aqueous vehicle, whereas those of a lipophilic nature will generally be present in the vesicular bilayer. The concentration of active agent in the vesicle phase is generally from 0.01 to 10% wt/wt.

The formulation is generally prepared by forming a mixture of the vesicle components—usually by melting these together and allowing to cool. In order to produce a vesicle suspension an aqueous liquid containing the active agent may be added to the melted vesicle formulation (e.g. at a temperature of 70° C.-100° C.) followed by vigorous agitation. The vesicle suspension may be extruded through a porous membrane to modify the particle diameter. The formulation may be used as produced, or the concentration of active agent in the aqueous phase may be varied as required. It has been found to be particularly advantageous for the active agent to be both entrapped by the vesicles and be present in the surrounding aqueous phase.

As discussed below, the non-ionic vesicle formulations of the present invention may be made with a variety of drugs, non-ionic surfactant vesicles, propellants, cosolvents, and other ingredients. Among the benefits provided by the invention, the vesicles may have enhance physical and biodegradation properties, function as a solubilising and/or chemical stabilising aid, provide sustained release.

The vesicle formulations according to the present invention contain a drug either dispersed, dissolved or otherwise entrapped by the vesicles and optionally or preferably also dissolved with the aqueous or solvent environment surrounding the vesicles. The drug is present in the formulation in a therapeutically effective amount (i.e. an amount suitable for the desired condition, route, and mode of administration). As used herein, the term “bioactive agent, includes its equivalents, “drug”, and “medicament” and is intended to have its broadest meaning as including substances intended for use in the diagnosis, cure, mitigation, treatment or prevention of disease, or to affect the structure or function of the body. The drugs can be neutral or ionic. Preferably, they are suitable for oral and/or nasal inhalation. Delivery to the respiratory tract and/or lung, in order to effect bronchodilation and to treat conditions such as lung cancer, asthma and chronic obstructive pulmonary disease, is preferably by oral inhalation. Alternatively, to treat conditions such as rhinitis or allergic rhinitis, delivery is preferably by nasal inhalation.

Any of a variety of therapeutic, prophylactic or diagnostic agents can be delivered. Examples include synthetic inorganic and organic compounds, proteins and peptides, polysaccharides and other sugars, lipids, and nucleic acid sequences having therapeutic, prophylactic or diagnostic activities. Nucleic acid sequences include genes, antisense molecules which bind to complementary DNA to inhibit transcription, and ribozymes. In some instances, the proteins may be antibodies or antigens which otherwise would have to be administered by injection to elicit an appropriate response. Compounds with a wide range of molecular weight can be encapsulated, for example, between 100 and 500,000 grams per mole.

Proteins are defined as consisting of 100 amino acid residues or more; peptides are less than 100 amino acid residues. Unless otherwise stated, the term protein refers to both proteins and peptides. Examples include insulin and other hormones. Polysaccharides, such as heparin, can also be administered.

Suitable drugs include, for example, anticancer agents, particularly cytotoxic agents, antiallergics, analgesics, bronchodilators, antihistamines, antiviral agents, antitussives, anginal preparations, antibiotics, antiinflammatories, immunomodulators, 5-lipoxygenase inhibitors, leukotriene antagonists, phospholipase A₂ inhibitors, phosphodiesterase IV inhibitors, peptides, proteins, steroids and vaccine preparations. A group of preferred drugs include adrenaline, albuterol, atropine, beclomethasone dipropionate, budesonide, butixocort propionate, clemastine, cromolyn, epinephrine, ephedrine, fentanyl, flunisolide, fluticasone, formoterol, ipratropium bromide, isoproterenol, lidocaine, morphine, nedocromil, pentamidine isoethionate, pirbuterol, prednisolone, sabutamol, salmeterol, terbutaline, tetracycline and pharmaceutically acceptable salts and solvates thereof, and mixtures thereof.

For oral and/or nasal inhalation, formulations where the drug is in solution and chemically stable are generally preferred; however, the drug may also be present in suspensions. Typically the solution will be an aqueous, pharmaceutically acceptable solution. However, where the drug displays poor solubility in an aqueous solution, other pharmaceutically acceptable solvents such as a glycol, alcohols, DMSO, acetic acid, alkyl acetate, ethyl ether etc., may be used. These may be present in the form of a co-solvent in an amount of 0.01% to 25% by weight of the total weight of the formulation. Alternatively or additionally agents which may improve the solubility of a particular drug in a chosen solvent, may be employed e.g. cyclodextrin specific pH or changing pH.

Preferably, vesicle formulations according to the present invention include a drug in an amount and in a form such that the drug can be administered as an aerosol. More preferably, the drug is present in an amount such that the drug can produce its desired therapeutic effect with one dose from a conventional aerosol canister with a conventional valve, such as a metered dose valve. As used herein, an “amount” of the drug can be referred to in terms of quantity or concentration. A therapeutically effective amount of a drug can vary according to a variety of factors, such as the potency of the particular drug, the route of administration of the formulation, the mode of administration of the formulation, and the mechanical system used to administer the formulation.

The formulations of the present invention are suitable for nasal and/or oral inhalation. By this it is meant, among other things, that when delivered from a metered dose inhaler they form particles of a size appropriate for nasal and/or oral inhalation and do not typically form films.

The formulations of the present invention may in certain embodiments possess sustained release properties.

A sustained release formulation is one that releases the drug over an extended period of time (e.g. as short as about 60 minutes or as long as several hours and even several days or months), rather than substantially instantaneously upon administration. Typically, for the vesicles as described herein, the sustained release characteristics are determined by the nature of the vesicle components and of the drug. Also, it is determined by the relative amount of vesicle components to drug.

A sustained release medicinal formulation includes drug entrapped vesicles in an amount such that the period of therapeutic activity of the drug is increased relative to the activity of the same formulation with respect to the propellant and drug but without the vesicles. When used in aerosol formulations, it will be understood by one of skill in the art that a direct comparison of the same formulation without the vesicle may not be possible due to formulation difficulties when the vesicles are absent. Thus, a conventional dispersant and/or cosolvent may need to be added to the medicinal formulation to provide an inhalable formulation for comparison of the period of time during which the drug is present at levels need to obtain a desired biological response. However, such formulation changes may prevent a perfectly parallel comparison of the release rates.

In a particularly preferred aspect, the formulations of the present invention are for use in treating lung cancer. Typically the active agent is a cytotoxic agent, such as a platinum based agent.

The various platinum-containing formulations are comprised of a platinum-based drug, such as cisplatin, and any stabilisers, together with non-ionic vesicles between about 10 nm and about 1000 microns preferably 15-1000 nm, more preferably 300-900 nm, or those greater than 1 micron in diameter, preferably 2-5 microns in diameter. Additionally, when desired, the platinum-containing formulation can contain additional components, such as transferrin or a platinum-transferrin complex, and use carriers, such as hydrofluorocarbons or fluorochlorocarbons (including 1,1,1,2,3,3,3-heptafluoropropane, 1,1,1,2-tetrafluoroethane, dichlorodifluoromethane, trichlorofluoromethane, or 1,2-dichloro-1,1,2,2-tetrafluoroethane), and/or excipients, such as sugars, including milk sugars such as lactose.

In addition or alternatively to cisplatin, other platinum-containing drugs that may be used in the formulation include one or more of: carboplatin, oxaliplatin, iproplatin, tetraplatin, transplatin, (cis-amminedichloro(cyclohexylamine)platinum(II)), (cis-amminedichloro(cyclohexylamine)-trans-dihydroxoplatinum(IV)), (bis-acetato-cis-amminedichloro(cyclohexylamine)platinum(IV)) and (trans-amminedichloro(cyclohexylamine)dihydroxoplatinum(IV)).

The formulations are used to treat lung cancers by inhalation of the anti-cancer such as platinum-containing formulations into the subject's respiratory tract. Lung cancers include both small cell and non-small cell primary lung cancer as well as cancers that metastasize to the lungs or the lung lymphatics. In addition, the invention describes methods of treating other cancers, such as bronchoalveolar carcinoma, leukaemia, myelomas, mesotheliomas, cancers of the bronchial pathways, trachea, or esophagus, and cancers of the liver or spleen, by inhalation of a platinum-containing formulation which will be cleared from the lungs via cellular uptake and transferred to the lymphatic system.

Inhalation is preferable to injection or infusion for three main reasons. First, it allows for localised administration of the antineoplastic agent to tumours of the bronchial pathways, lungs, and surrounding tissues. Localised administration has been shown to increase the effectiveness of platinum-containing drugs on other types of cancers. The therapeutic index of the drug will be greatly enhanced due to lower dose needed, systemic by-pass, and targeting to the affected cells. Second, subjects generally prefer inhalation to injection or infusion because it is less invasive and will cause fewer unpleasant systemic side effects. By avoiding wide-spread dispersions throughout the body, as occurs with intravenous use, fewer non-cancerous cells will be exposed to the toxic effects of the drug, and therefore, the subject will experience less nausea and vomiting and be at less of a risk for kidney damage or hearing loss. Third, treatment by inhalation will likely be less costly than treatment by infusion because it is easier to administer. In appropriate circumstances, subjects could receive treatment in their own homes, possibly even by self-administration.

### BRIEF DESCRIPTION OF THE FIGURES

FIG. 1 is a series of charts showing shows the effect of treatment of BALB/c mice by inhalation with free or vesicular amphotericin-B (“NIV”) on drug levels (μg/ml+SE) in the lungs, serum and lung lavage.

FIGS. 2a-d are a series of photomicrographs showing the appearances of different cisplatin-NIV formulations formed by hydrating with 0.5 mg/ml cisplatin (a), 6 mg/ml cisplatin (b), 6 mg/ml cisplatin then diluted with saline to give 0.5 mg/ml cisplatin (c) or hydrated with 6 mg/ml cisplatin, diluted with saline to give 0.5 mg/ml cisplatin, and then processed to increase the vesicle content within the suspension (‘Processed cisplatin-NIV).

### DETAILED DESCRIPTION

The present invention will now be further described by way of example and with reference to the Figures which show:

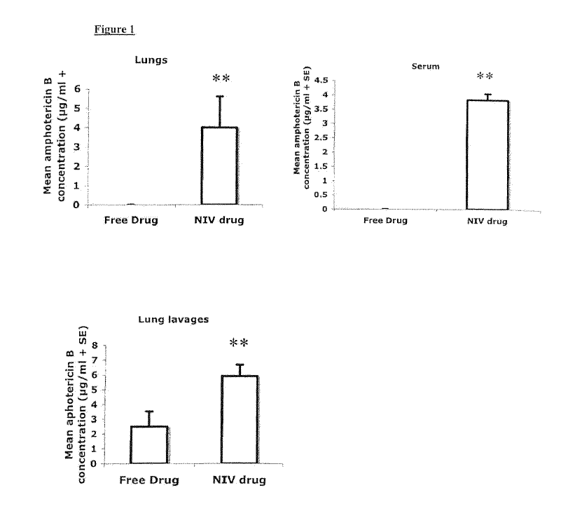

FIG. 1 shows the effect of treatment by inhalation with free or vesicular amphotericin-B on drug levels in the lungs, serum and lung lavage. BALB/c mice were exposed for 20 minutes by inhalation to free amphotericin B solution (1 mg/ml) or amphotericin B-NIV (1 mg/ml) on days 1, 2 and 3 using a Buxco® nebulisation system and the mass dosing box supplied. Amphotericin B solution at 1 mg/ml was obtained by using cyclodextrin to solubilise the drug using the method described by Mullen et al., (1997). 15 minutes after dosing the animals were sacrificed and drug levels in tissues were determined by HPLC. Lung lavages were collected from mice at sacrifice using two washes of 0.8 ml phosphate buffered saline, pH 7.4 (PBS). Four mice were used/treatment. **p<0.01 compared to free drug treatment.

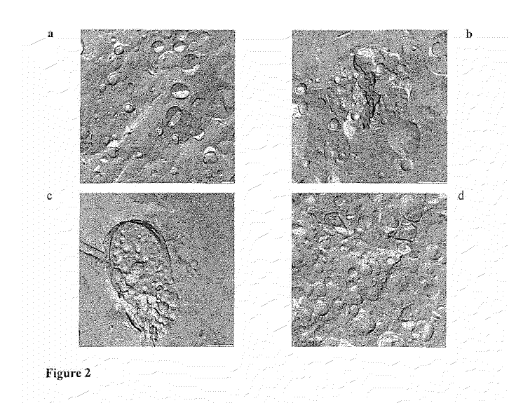

FIG. 2 Appearance of different cisplatin-NIV formulation. The NIV were formed by hydrating with 0.5 mg/ml cisplatin (a), 6 mg/ml cisplatin (b), 6 mg/ml cisplatin then diluted with saline to give 0.5 mg/ml cisplatin (c) or hydrated with 6 mg/ml cisplatin, diluted with saline to give 0.5 mg/ml cisplatin, and then processed to increase the vesicle content within the suspension (‘Processed cisplatin-NIV).

### EXAMPLES SECTION

### Ready-to Use Formulation

3000 or 600 μmol vesicle constituents, consisting of 3:3:1 molar ratio of mono-n-hexadecyl ether tetraethylene glycol, cholesterol and dicetyl phosphate were melted by heating at 130° C. for 5 minutes. The molten mixture was cooled to 70° C., and hydrated with 5 ml of preheated (70° C.) distilled water or drug solution (NIV). Vesicular formulations were homogenised at 8000±100 rpm for 15 minutes at 70° C., using a Silverson mixer, Model L4R SU (Silverson Machines, UK), fitted with a ⅝″ tubular work head. NIV drug suspensions can be diluted with saline or distilled water to a set drug concentration/ml prior to use. These NIV suspensions are stored as appropriate before use.

### Processed NIV

In some cases NIV suspensions were processed to increase the concentration of vesicles/ml in the formulation. 3000 or 600 μmol vesicle constituents, consisting of 3:3:1 molar ratio of mono-n-hexadecyl ether tetraethylene glycol, cholesterol and dicetyl phosphate were melted by heating at 130° C. for 5 minutes. The molten mixture was cooled to 70° C., and hydrated with 5 ml of preheated (70° C.) distilled water or drug solution (NIV). Vesicular formulations were homogenised at 8000±100 rpm for 15 minutes at 70° C., using a Silverson mixer, Model L4R SU (Silverson Machines, UK), fitted with a ⅝″ tubular work head. NIV drug suspensions can be diluted with saline or distilled water to a set drug concentration/ml prior to use. Suspensions were then passed through a Vivaflow 50 cartridge (Sartorius Stedi, UK Ltd., Epson, UK) to concentrate NIV without altering the concentration of un-entrapped drug, these NIV formulations were known as “processed-NIV”. The final volume depends on requirements and is limited by the ‘dead volume’ of the cartridge which for the Vivaflow 50 is approximately 5-10 mls.

For example processed cisplatin-NIV were formed by heating, a 3:3:1 molar ratio of mono-n-hexadecyl ether tetraethylene glycol, cholesterol and dicetyl phosphate (3000 μM lipid) at 130° C. for 5 minutes and then cooling the molten mixture 70° C. The mixture was then hydrated with 5 ml of preheated (70° C.) cisplatin at 6 mg/ml and the resulting suspension was homogenised at 8000±100 rpm for 15 minutes at 70° C., using a Silverson mixer, Model L4R SU (Silverson Machines, UK), fitted with a ⅝″ tubular work head. The suspension was then diluted with saline to 0.5 mg cisplatin/ml by adding 55 mls saline. Suspensions were then passed through a Vivaflow 50 cartridge (Sartorius Stedi, UK Ltd., Epson, UK) to concentrate the NIV to give a final volume of 6.4 mls, which would concentrate the formulation by a factor of 9.4 concentration, giving a final lipid concentration of 94 μM/ml lipid

### Freeze-Dried Formulations

3000 or 600 μmol vesicle constituents, consisting of 3:3:1 molar ratio of mono-n-hexadecyl ether tetraethylene glycol, cholesterol and dicetyl phosphate were melted by heating at 130° C. for 5 minutes. The molten mixture was cooled to 70° C., and hydrated with 5 ml of preheated (70° C.) distilled water. Vesicular formulations were homogenised at 8000±100 rpm for 15 minutes at 70° C., using a Silverson mixer, Model L4R SU (Silverson Machines, UK), fitted with a ⅝″ tubular work head. Suspensions were stored at −80° C. and then lyophilised before storage at −20° C. The lyophilised NIV were hydrated with drug solution just before use. These formulations may be processed as described above so that they contained a higher concentration of vesicles/ml.

### Animals

Age and gender matched BALB/c mice (20-25 g, male or female) in-house bred at Strathclyde University or purchased form Harlan Olac (Bicester, UK) were used in this study. Commercially obtained Golden Syrian hamsters (Mesocricetus auratus, Harlan Olac, Bicester, UK) were used for maintenance of L. donovani (parasite strain LV82; Carter et al., 1988). Animal experiments were carried out in accordance with UK Home Office regulations.

### Determination of Drug Levels

Mice were treated with the appropriate drug formulation either by intravenous injection of 0.2 ml of the appropriate drug formulation or PBS pH 7.4/saline (controls) or by inhalation with the appropriate drug formulation or saline (controls) using the Buxco® nebulisation system. 0.5 ml of the appropriate formulation or saline was added to the nebuliser which was attached to a Volumatic™ Spacer (Allen and Hanbury, Uxbridge, Middlesex). After 1.5 mins of nebulisation, the nebuliser was switched off, and the mice were exposed to the resulting aerosol for a further 5 minutes. 5 minutes after dosing mice were injected intraperitoneally with a general anaesthetic. Blood was taken by cardiac puncture and allowed to clot by storing for up to 12 hours at 4° C. Serum was separated from clotted blood sample by centrifuging at 13,000 rpm at 4° C., and transferred to a clean eppendorf tube. The lungs of the mouse were inflated by introducing two samples of 0.8 ml PBS pH 7.4 via the trachea and the resulting lung lavages were transferred to a clean eppendorf tube. Various organs were removed from the mouse and stored in 1.5 ml microfuge tubes. All the samples were stored at −20° C. until cisplatin levels within the samples could be determined using an HPLC assay.

In Vivo Efficacy Studies Using Leishmania donovani

BALB/c mice were infected by intravenous injection (tail vein, no anaesthetic) with 1-2×10⁷ L. donovani amastigotes (Carter et al., 1988). The day of parasite administration to the mice was designated day 0 of the experiment. On day 7 post-infection mice were treated by intravenous injection with 0.2 ml of the appropriate formulation by intravenous injection, without anaesthetic (n=4/treatment). On day 14 impression smears of the spleen and liver, and a smear of bone marrow, were made on to an individual glass microscope slide for each mouse at sacrifice. The slides were fixed in methanol for 30 secs, stained in 10% aqueous Giemsa stain (BDH, VWR International Ltd., Poole, UK) for 20 minutes, and then allowed to air dry. The number of parasites present/1000 host nuclei for the spleen, liver and bone marrow for each sample was determined at ×1000 magnification. The LDU was calculated by multiplying the number of parasites present/1000 host nuclei by the organ weight (gms) for the spleen and liver.

### In Vivo Lung Cancer Efficacy Studies

BALB/c mice (n=8/treatment) were injected intravenously on day 0 with 1-5×10⁵ B16 F0 cells which form metastatic lung and liver tumours in mice (Nakurmura et al., 2002). On day 7 mice were treated either by intravenous injection of 0.2 ml of the appropriate drug formulation or PBS pH 7.4/saline (controls) or by inhalation with the appropriate drug formulation or saline using the Buxco® nebulisation system. 0.5 ml of the appropriate formulation or saline was added to the nebuliser which was attached to a Volumatic™ Spacer (Allen and Hanbury, Uxbridge, Middlesex). After 1.5 mins of nebulisation the nebuliser was switched off and the mice were exposed to the resulting aerosol for a further 5 minutes. Mice were sacrificed on day 14, body weight and the weight of the spleen, liver, lungs and kidney assessed. Lung weight was taken as an indirect measure of tumour burden. In some cases mice were dosed on days 4, 7, 9 and 11. Tumours were easily visible as black areas on the surface of the lungs and liver.

### Vesicle Characteristics

Vesicle size (mean diameter±SD) depended on the moiety entrapped and freeze-dried NIV hydrated with a moiety just before use were larger than the same formulation produced as a ‘ready to use’ preparation. For example, rifampicin-NIV hydrated with rifamapicin/cyclodextrin solution (20 mg/ml) had a mean diameter of 361±16 nm, whereas freeze-dried NIV reconstituted with the same drug solution had a mean diameter of 531±54 nm. Cisplatin-NIV, prepared using 6 mg/ml, had a mean diameter of 1629±84 nm. Subsequent dilution of this drug formulation to 0.5 mg/ml with saline and concentration using the Vivaflow 50 system produced NIV with a mean diameter of 1733±363 nm. Cisplatin-NIV, prepared using 0.5 mg/ml, had a mean diameter of 542±24 nm. When nebulised the vesicles will be delivered in droplets (approxaimate 2-5 μm) produced by the nebulsisation system used to produce an aerosol.

### Example 1

### Rifampicin

Rifampacin is an antibiotic which is used to treat tuberculosis (TB) infections but it can be used to treat other infections e.g. aerobic gram negative bacteria, Neisseria meningitidis. It has been shown to be hepatotoxic and most treatments are prolonged and can last up to 6-12 months. The actual mechanism for liver toxicity is not understood but the formation of toxic metabolites is important (Tostmann et al., 2008). Ideally shorter treatment regimens with lower toxicity are required. The ability to treat TB by inhalation so that the drug is targeted to the organism within the lungs, whilst reducing exposure of the liver, would allow a reduction in drug dose and/or number of doses used, and reduce potential toxicity of the drug.

No drug was detected in any of the samples taken from mice given one dose of free or vesicular rifampicin by inhalation, whereas treatment with the oral commercial formulation resulted in drug levels in the serum and lung lavages but not the lungs (Table 1). In contrast, treatment with three doses by inhalation resulted in drug being found in all three samples taken from mice given any of the formulations (Table 1). Treatment with the vesicular formulation resulted in significantly (p<0.01) higher drug levels in the lungs compared to the other two formulations used (Table 1). There was no significant difference in the drug levels present in the serum or lung lavages of mice treated with three doses of the different rifampicin formulations (Table 1).

Using a Volumatic™ spacer to administer the drug, resulted in better tissue loading, presumably the smaller volume of the receptacle containing the mice meant they were exposed to higher local concentration of the drug formulation (compare Tables 1 and 2). Significantly (p<0.01) higher levels of rifampicin were found in the lungs and lung lavages of animals given rifampicin-NIV compared to treatment with free rifampicin given by inhalation or oral treatment with Rifadin® (a commercial oral formulation of rifampicin).

TABLE 1
Rifampicin drug levels in mice treated with different rifampicin
drug formulations

| | Mean drug concentration | | |
|---|---|---|---|
| | (μg/ml ± SE) | | |
| | | | Lung |
| Treatment | Serum | Lungs | lavages |
| Expt 1 | | | |
| 1 dose Free rifampicin | nd | nd | nd |
| inhalation | | | |
| 1 dose Rifampicin- | nd | nd | nd |
| NIV inhalation | | | |
| 1 doses Rifadin ® | 1.91 ± 0.62 | nd | 0.82 ± 0.11 |
| Expt 2 | | | |
| 3 doses Free | 19.6 ± 1.02 | 0.54 ± 0.14 | 2.16 ± 0.84 |
| rifampicin | | | |
| 3 doses Rifampicin- | 16.32 ± 4.39 | 1.55 ± 0.79** | 1.31 ± 0.46 |
| NIV | | | |
| 3 doses Rifadin ® | 9.00 ± 1.2 | 0.45 ± 0.07 | 0.61 ± 0.10 |

BALB/c mice were exposed for 20 minutes by inhalation using a Buxco ® nebulisation system and the mass dosing box supplied with free rifampacin solution (20 mg/ml) or rifampacin-NIV (20 mg/ml) on day 1 only (expt 1) or days 1, 2 and 3 (Expt 2). A group of mice were also orally treated with Rifadin ®, a commercial oral formulation of rifampicin, at a dose of 20 mg/kg/dose using the same dosing regimen. 15 minutes after dosing, the animals were sacrificed and drug levels in tissues were determined. Lung lavages were collected from mice at sacrifice using two washes of 0.8 ml phosphate buffered saline, pH 7.4 (PBS). Rifampicin levels were determined using an HPLC assay. Six mice were used/treatment.
**p < 0.01 compared to free drug treatment.

TABLE 2
Rifampicin drug levels in mice treated with different rifampicin
drug formulations.

| | Mean rifampicin | | |
|---|---|---|---|
| | concentration (μg/ml) | | |
| Treatment | Serum | Lung lavages | Lungs |
| Rifadin ® oral | 2.26 ± 1.41** | 0.54 ± 0.13** | 0.00 ± 0.00*** |
| Free rifampicin | 5.23 ± 2.50 | 1.00 ± 0.21* | 0.47 ± 0.18*** |
| inhalation | | | |
| Rifampicin-NIV | 15.21 ± 2.14 | 1.72 ± 0.20 | 1.19 ± 0.24 |
| inhalation | | | |

BALB/c mice were exposed for 6.5 minutes by inhalation to free rifampacin solution (20 mg/ml) or rifampacin-NIV (20 mg/ml) on days 1, 2 and 3 using a Buxco ® nebulisation system attached to a Volumatic ™ spacer. A group of mice were also orally treated with Rifadin ®, a commercial formulation of rifampicin, at a dose of 20 mg/kg/dose using the same dosing regimen. 15 minutes after dosing, the animals were sacrificed and drug levels in tissues were determined using an HPLC assay. Lung lavages were collected from mice at sacrifice using two washes of 0.8 ml phosphate buffered saline, pH 7.4 (PBS). Rifampicin levels were determined using an HPLC assay. Six mice were used/treatment.
*p < 0.05 compared to rifampicin-NIV treatment,
**p < 0.01 compared to rifampicin-NIV treatment,
***p < 0.001 compared to rifampicin-NIV treatment.

### Example 2

### Amphotericin B

Amphotericin B is a macrolide antibiotic derived from Streptomyces nodosus. It is used in the treatment of systemic fungal infections and its selectivity is based on its ability to target 24-substituted sterols such as ergosetrol or episterol found in fungal cells instead of cholesterol, which is found in mammalian cell membranes. Its use is associated with severe side effects such as anaphylaxis, cardiac arrythmias, anaemia and nephrotoxicity (Mullen et al., 1997). This has led to the development of lipid formulations, which give better drug targeting so that a lower drug dose/number of doses can be used for this drug, which has low aqueous solubility. Development of better Amphotericin B drug formulations would be useful especially if they could target the lungs and minimise liver exposure. This could be particularly helpful in the management of invasive pulmonary aspergillus infections, which occur in 1.5-10% of liver transplant patients, partly because of the immunosuppressive agents given to patients, and can be life threatening (Takeda et al., 2007). Fungal infections can occur in immunosuppressed patients, either as a result of giving immunosuppressive drugs as part of the treatment to manage another condition, or as part of the disease symptom e.g. AIDS patients.

Treatment with amphotericin B-NIV resulted in significantly higher drug levels in the lungs, serum and lung lavages of mice compared to similar treatment with free drug solution (FIG. 1).

### Example 3

### Cisplatin

Cisplatin is a cytotoxic drug used in the treatment of cancer. It is poorly absorbed and only 0.1% of the dose reaches the lungs after intravenous administration, where it has a short residence time. This means that patients require multiple doses of the drug to have a therapeutic effect but its toxic side effects means that the drug has to be given in cycles with drug free periods, to allow the patient to recover. Nephrotoxicty and neurootoxicity are the main side effects of the drug (Hanigan and Devarajan, 2003) and novel formulations with reduced toxicity compared to cisplatin solution and similar or better therapeutic efficacy are required.

Treatment by intravenous injection with cisplatin NIV hydrated with 0.5 mg/ml (formulation 1, Table 3) resulted in significantly higher drug levels in the spleen, liver and kidneys but was not associated with an increase in lung levels. The effect of increasing the cisplatin concentration used to hydrate NIV was determined but the cisplatin solution had to be heated as maximum aqueous solubility of cisplatin is approximately 1 mg/ml at room temperature. Therefore, NIV were hydrated with a 6 mg/ml cisplatin solution at 70° C. and after homogenisation the suspension was diluted to 0.5 mg/ml with saline (formulation 2, Table 3) so that the cisplatin did not crystallise out of solution. Intravenous treatment with this formulation gave significantly higher drug levels in the spleen and liver, but not the kidney. However, this formulation did not enhance drug delivery to the lungs. Although treatment by inhalation with the NIV formulations increased drug delivery to the spleen and liver, with NIV hydrated with the higher drug concentration being more effective, there was no appreciable drug delivery to the lungs (Table 3). Intravenous treatment with free cisplatin at 4.44 mg/kg had no effect on tumour growth (Table 5). In contrast exposure to free cisplatin for 6.5 minutes by inhalation resulted in a significant reduction in lung weight, which was taken as an indirect measure of tumour growth, compared to control values (Table 5). This effect was obtained even though no cisplatin was detected 5 minutes after free drug treatment by inhalation (Table 3), indicating that very little drug is required in the lungs to have an anti-cancer effect or that the drug has a very short residence time in the lungs after dosing.

As hydrating NIV with cisplatin at a drug concentration above its solubility required a subsequent dilution step to ensure that any un-entrapped drug did not precipitate prior to removal using a Vivaflow 50 system. It would be possible to process the cisplatin-NIV formulation to remove all entrapped drug using this system, but previous had studies shown that suspending drug loaded NIV in a drug solution helped to maintain drug levels within the NIV and preserve their in vivo efficacy (Patent, WO 96/04890). Treatment by intravenous injection with the processed NIV formulation resulted in significantly higher drug levels in all samples with the greatest enhancement being obtained for the lungs and liver (Table 4). However, delivery of the processed NIV by this route also resulted in higher levels in the kidney. In contrast treatment by inhalation with the processed formulation only resulted in a higher drug levels in the lungs (Table 4). Processing the NIV would have increased the NIV content of the suspension by a factor of 4 based on lipid content. However delivery using this formulation caused an even greater increase in tissue drug levels which was more apparent by the intravenous route. For example, no drug was found in the lung levels in mice treated with NIV formulation 1, intravenous treatment with the processed NIV increased drug levels by a factor of 808 whereas treatment by inhalation increased drug levels by a factor of 16.

Data from ultrastructural studies suggests that processing changes the characteristics of the vesicles and could possibly prevent the formation of cisplatin crystals in NIV on cooling or affect the physical characteristics of the vesicles if they had multilamellae (see FIG. 2).

Treatment by inhalation with 4 doses of the processed cisplatin NIV resulted in a similar significant reduction in lung weight as that caused by free cisplatin treatment (p<0.01, Expt 1, Table 6). However a differential effect compared to free drug treatment was only apparent if the processed cisplatin-NIV formulation was diluted 1:5 before use. In this case treatment with the diluted processed cisplatin-NIV formulation caused a significant reduction in lung weight compared to control values (p<0.05) whereas similar treatment with free cisplatin had no significant effect on lung weight (Expt 2, Table 6). Diluting the processed cisplatin-NIV formulation 1:10 before use resulted in a decrease in efficacy as lung weights were similar to control values. This is not unexpected as treatment with the processed cisplatin-NIV formulation resulted in a 16-fold increase in cisplatin lung levels (Table 5), indicating that a 10 fold dilution in the formulation would not give appreciably higher cisplatin levels at this site.

TABLE 3
Cisplatin levels in sample from mice treated by inhalation or
intravenous injection with different cisplatin formulations.

| | | Mean cisplatin | Fold change |
|---|---|---|---|
| | | concentration | compared |
| | | (μg/ml or | to free drug |
| Treatment | Sample | μg/ml/g tissue ± SE) | treatment |
| Cisplatin | Serum | 0.296 ± 0.13 | |
| solution iv | Spleen | 0.065 ± 0.02 | |
| n = 5 | Liver | 0.19 ± 0.13 | |
| | Lungs | 0 ± 0 | |
| | Lung lavages | 0.13 ± 0.07 | |
| | Kidney | 0.951 ± 0.35 | |
| Cisplatin-NIV | Serum | 0.336 ± 0.16 | 1.1 |
| formulation 1 iv | Spleen | 1.79 ± 1.68 | 27.5 |
| n = 5 | Liver | 0.468 ± 0.24 | 2.5 |
| | Lungs | 0 ± 0 | 0 |
| | Lung lavages | 0.113 ± 0.11 | 0.9 |
| | Kidney | 4.60 ± 1.69 | 4.8 |
| Cisplatin-NIV | Serum | 0.274 ± 0.14 | 0.9 |
| formulation 2 iv | Spleen | 0.949 ± 0.50 | 14.6 |
| n = 5 | Liver | 0.763 ± 0.42 | 4.0 |
| | Lungs | 0 ± 0 | 0 |
| | Lung lavages | 0.001 ± 0.001 | 0.01 |
| | Kidney | 0.881 ± 0.50 | 0.9 |
| Cisplatin | Serum | 0.268 ± 0.1 | |
| solution | Spleen | 0.018 ± 0.01 | |
| inhalation | Liver | 0.037 ± 0.04 | |
| n = 5 | Lungs | 0 ± 0 | |
| | Lung lavages | 0.004 ± 0.002 | |
| | Kidney | 6.81 ± 2.53 | |
| Cisplatin-NIV | Serum | 0.058 ± 0.002 | 0.2 |
| formulation 1 | Spleen | 0.058 ± 0.002 | 3.2 |
| inhalation | Liver | 0.008 ± 0.005 | 2.2 |
| n = 5 | Lungs | 0 ± 0 | 0 |
| | Lung lavages | 0.008 ± 0.002 | 8 |
| | Kidney | 0.062 ± 0.03 | 0.01 |
| Cisplatin-NIV | Serum | 0.142 ± 0.08 | 0.5 |
| formulation 2 | Spleen | 0.288 ± 0.26 | 16 |
| inhalation | Liver | 0.036 ± 0.03 | 1.0 |
| n = 5 | Lungs | 0 ± 0 | 0 |
| | Lung lavages | 0.002 ± 0.001 | 0.5 |
| | Kidney | 0.096 ± 0.03 | 0.01 |

BALB/c mice were exposed for 6.5 minutes to the drug formulations by inhalation or treated with 0.2 ml of the appropriate formulation by intravenous injection, without an anaesthetic. Drug formulations given by inhalation were nebulised directly into the Spacer for 1.5 minutes using a Buxco ® nebulisation system which delivered 0.5 ml of the appropriate formulation into the Volumatic ™ Spacer and then maintained in that environment for a further 5 minutes. Mice were treated with free cisplatin (0.5 mg/ml), cisplatin-NIV formulation 1 (300 μM lipid hydrated with 5 mls of cisplatin solution at 0.5 mg/ml) or cisplatin-NIV formulation 2 (3000 μM lipid hydrating with 5 mls of cisplatin at 6 mg/ml and then diluted to 0.5 mg/ml with saline just before use). 5 minutes after dosing the animals were sacrificed and drug levels in tissues were determined. Lung lavages were collected from mice at sacrifice using two washes of 0.8 ml PBS, pH 7.4. Cisplatin levels were determined using an HPLC assay. Drug levels for serum and lung lavages are in μg/ml whereas levels for the other samples, i.e. spleen, liver, lungs, kidney, are expressed as μg/ml/g tissue. The ability of the NIV to target the drug to tissues is shown by the fold change in drug levels compared to free drug levels obtained using the same administration route, calculated using mean values shown in the Table.

TABLE 4
Cisplatin levels in sample from mice treated by inhalation or
intravenous injection with different cisplatin formulations.

| | | Mean cisplatin | |
|---|---|---|---|
| | | concentration | Fold change |
| | | (μg/ml or | compared to free |
| Treatment | Sample | μg/ml/g tissue ± SE) | drug treatment |
| Cisplatin | Serum | 0.207 ± 0.02 | |
| solution iv | Spleen | 0.346 ± 0.10 | |
| n = 5 | Liver | 0.032 ± 0.02 | |
| | Lungs | 0.032 ± 0.015 | |
| | Lung lavages | 0.095 ± 0.06 | |
| | Kidney | 0.55 ± 0.48 | |
| Processed | Serum | 0.806 ± 0.11 | 3.9 |
| cisplatin-NIV iv | Spleen | 20.93 ± 2.75 | 60.5 |
| n = 5 | Liver | 9.35 ± 1.58 | 292.2 |
| | Lungs | 25.87 ± 4.18 | 808.4 |
| | Lung lavages | 0.202 ± 0.03 | 2.2 |
| | Kidney | 17.20 ± 7.19 | 31.3 |
| Cisplatin | Serum | 0 ± 0 | |
| solution | Spleen | 0.346 ± 0.19 | |
| inhalation | Liver | 0.032 ± 0.02 | |
| n = 5 | Lungs | 0.032 ± 0.15 | |
| | Lung lavages | 0.095 ± 0.06 | |
| | Kidney | 0.55 ± 0.48 | |
| Processed | Serum | 0 ± 0 | 0 |
| cisplatin-NIV | Spleen | 0.083 ± 0.05 | 0.2 |
| inhalation | Liver | 0.041 ± 0.02 | 1.3 |
| n = 5 | Lungs | 0.52 ± 0.28 | 16.3 |
| | Lung lavages | 0.047 ± 0.019 | 0.5 |
| | Kidney | 0.04 ± 0.02 | 0.07 |

BALB/c mice were exposed for 6.5 minutes to the drug formulations by inhalation or treated with 0.2 ml of the appropriate formulation by intravenous injection, without an anaesthetic. Drug formulations given by inhalation were nebulised directly into the Spacer for 1.5 minutes using a Buxco ® nebulisation system which delivered 0.5 ml of the appropriate formulation into the Volumatic ™ Spacer and then manintained in that environment for a further 5 minutes. Mice were treated with free cisplatin (0.5 mg/ml) or a processed cisplatin-NIV formulation (3000 μM lipid, prepared by hydrating with 6 mg/ml and then diluted to 0.5 mg/ml with saline and then processed using a Vivaflow 50 cartridge so that the final drug concentration, based on un-entrapped drug, was 0.5 mg/ml and the lipid concentration was approximately 2125 μM). 5 minutes after dosing the animals were sacrificed and drug levels in tissues were determined. Lung lavages were collected from mice at sacrifice using two washes of 0.8 ml PBS, pH 7.4. Cisplatin levels were determined using an HPLC assay. Drug levels for serum and lung lavages are in μg/ml whereas levels for the other samples, i.e. spleen, liver, lungs, kidney, are expressed as μg/ml/g tissue. The ability of the NIV to target the drug to tissues is shown by the fold change in drug levels compared to free drug levels obtained using the same administration route, calculated using mean values shown in the Table.

TABLE 5
The effect of treatment with cisplatin formulation by injection or
inhalation on the growth of B10 F0 cells in BALB/c mice

| | Mean organ weight | | | |
|---|---|---|---|---|
| | (g ± SE) | | | |
| Treatment | Lungs | Liver | Spleen | Kidney |
| IV control | 0.36 ± 0.02 | 1.55 ± 0.08 | 0.23 ± 0.01 | 0.13 ± 0.01 |
| Free cisplatin 4.44 mg/ | 0.42 ± 0.03 | 1.44 ± 0.16 | 0.22 ± 0.01 | 0.13 ± 0.01 |
| kg iv | | | | |
| Inhalation control | 0.42 ± 0.01 | 1.50 ± 0.08 | 0.23 ± 0.01 | 0.12 ± 0.01 |
| Free cisplatin | 0.22 ± 0.01*** | 1.47 ± 0.05 | 0.24 ± 0.01 | 0.19 ± 0.04 |
| 0.5 mg/ml | | | | |
| inhalation | | | | |
| Cisplatin-NIV | 0.23 ± 0.02*** | 1.53 ± 0.03 | 0.21 ± 0.01 | 0.11 ± 0.004 |
| 0.5 mg/ml | | | | |
| inhalation | | | | |

Mice were injected on day 0 with 2.7 × 10⁵ B16 F0 cells. On day 7 mice were treated either by intravenous injection with 0.2 ml saline (IV control) or cisplatin solution (0.5 mg/ml) or treated by inhalation by exposing for 6.5 mins to 0.5 ml of saline (Inhalation control), cisplatin solution (0.5 mg/ml) or cisplatin-NIV (hydrated with 0.5 mg/ml). Drug formulations given by inhalation were nebulised directly into the Spacer for 1.5 minutes using a Buxco ® nebulisation system which delivered 0.5 ml of the appropriate formulation into the Volumatic ™ Spacer and then manintained in that environment for a further 5 minutes. Mice were sacrificed on day 14 and organs weights determined.
***p < 0.001 compared to inhalation control.

TABLE 6
The effect of treatment with cisplatin formulation by injection or inhalation on the
growth of B10 F0 cells in BALB/c mice

| | Mean organ weight (g ± SE) | | | |
|---|---|---|---|---|
| Treatment | Lungs | Liver | Spleen | Kidney |
| Expt 1 | | | | |
| Inhalation control | 0.38 ± 0.02 | 1.14 ± 0.03 | 0.12 ± 0.004 | 0.16 ± 0.004 |
| Free cisplatin | 0.21 ± 0.01** | 1.19 ± 0.07 | 0.13 ± 0.01 | 0.15 ± 0.01 |
| Processed | 0.21 ± 0.01** | 0.88 ± 0.12 | 0.08 ± 0.004 | 0.12 ± 0.04 |
| cisplatin-NIV | | | | |
| Expt 2 | | | | |
| Inhalation control | 0.23 ± 0.01 | 1.54 ± 0.10 | 0.14 ± 0.01 | 0.25 ± 0.01 |
| Free cisplatin | 0.21 ± 0.01 | 1.30 ± 0.03 | 0.13 ± 0.01 | 0.21 ± 0.01 |
| 0.1 mg/ml | | | | |
| Processed | 0.19 ± 0.01* | 1.34 ± 0.08 | 0.11 ± 0.01* | 0.20 ± 0.01 |
| cisplatin-NIV 1:5 | | | | |
| Processed | 0.21 ± 0.01 | 1.35 ± 0.03 | 0.13 ± 0.01 | 0.21 ± 0.01 |
| cisplatin-NIV 1:10 | | | | |

Mice were injected on day 0 with 2.2 × 10⁵ B16 F0 cells. Mice were treated by inhalation by exposing for 6.5 mins to 0.5 ml of saline (Inhalation control), cisplatin solution (Expt 1 0.5 mg/ml; Expt 2 0.1 mg/ml) or processed cisplatin-NIV (hydrated with 6 mg/ml cisplatin, diluted to 0.5 mg/ml and then processed to increase vesicle content). Mice were dosed on days 4, 7, 9 and 11 in Expt 1 and on day 7 only in Expt 2. Drug formulations given by inhalation were nebulised directly into the Spacer for 1.5 minutes using a Buxco ® nebulisation system which delivered 0.5 ml of the appropriate formulation into the Volumatic ™ Spacer and then manintained in that environment for a further 5 minutes. In expt 1 processed cisplatin-NIV were used as prepared whereas in expt 2 the processed cisplatin-NIV were diluted with 1:5 or 1:10 with saline just before use. Mice were sacrificed on day 14 and organs weights determined.
**p < 0.01 compared to control,
*p < 0.05 compared to control.

In comparison to injection or infusion, the administration of a drug by inhalation is attractive. For some cancers, inhalation can provide a more localised administration of the therapeutic agent and, therefore, can be more effective. The increased effectiveness of local administration will be seen most in the lungs and bronchial pathways, but as the platinum-containing drug is cleared from the lungs via cellular uptake and transfer to the lymphatic system, it can act on cancers affecting other areas, such as the liver, spleen, and bone marrow.

With this local application approach, inhalation can reduce the side effects of cisplatin and other platinum-containing agents normally encountered after intravenous administration, due to limited bioavailability to tissues and organs via the blood stream. It can also be easier to administer therapeutics by inhalation. Cisplatin when administered intravenously is rapidly bound to various proteins found in the blood plasma, thus inactivating most of the intact platinum compound.

It may be preferably to formulate Cisplatin-NIV using saline. The inventors have observed, data not shown, that a carbonate buffer may interact with the cisplatin thereby reducing its efficacy.

### Example 4

### Drug Loading NIV Must be Maintained in a Drug Solution in Order to Maintain their In Vivo Efficacy

Treatment of L. donovani infected mice with free cisplatin at a dose of 4.44 mg/kg (or 14.4 mg/m², calculated using http://www.fda.gov/cder/cancer/animalframe.htm) had no significant effect on parasite liver burdens. However treatment with cisplatin NIV formulation A, prepared using 6 mg/ml cisplatin and then diluted to the same drug concentration of 0.5 mg/ml, resulted in a significant suppression in parasite liver burdens compared to control values (45% reduction based on mean values, Table 7). Therefore the reduction is parasite burdens could be attributed to the anti-leishmanial activity of the drug entrapped within NIV and not the influence of the un-entrapped drug solution. Two 1 ml samples of formulation A were submitted to ultracentrifugation and the resulting NIV pellet was resuspended in either cisplatin drug solution (0.5 mg/ml, cisplatin-NIV formulation B) or saline (cisplatin-NIV formulation C) to determine whether the solution which contained the drug loaded NIV had any influence on their in vivo efficacy. Removal of un-entrapped drug ablated the activity of cisplatin-NIV formulation as treatment cisplatin-NIV formulation C was unable to significantly reduce parasite liver burdens. In contrast, if the drug-loaded NIV were resuspended in cisplatin drug solution, then the NIV formulation (formulation B was as active as the parent formulation (formulation A, Table 7). Presumably removal of un-entrapped drug favoured cisplatin loss from the cisplatin-loaded NIV.

TABLE 7
The effect of treatment with different cisplatin formulation
on L. donovani liver parasite burdens in different tissues.

| Treatment | Mean number of parasites ± SE |
|---|---|
| Control | 2076 ± 332 |
| Free cisplatin | 2142 ± 249 |
| Cisplatin-NIV formulation A | 1140 ± 192^{a} |
| Cisplatin-NIV formulation B | 1156 ± 190^{a} |
| Cisplatin-NIV formulation C | 1889 ± 282^{b} |

Leishmania donovani infected mice were treated on day 7 post-infection without aneasthetic with 0.2 ml PBS pH 7.4 (control), cisplatin solution (0.5 mg/ml, 4.44 mg/kg), cisplatin formulation A (prepared by hydrating 3000 μM lipid with 5 mls 6 mg/ml cisplatin, diluted 1:12 to 0.5 mg/ml cisplatin just prior to injection), formulation B (hydrated with 6 mg/ml cisplatin, NIV were pelleted by centrifugation at 60,000 rpm for 2 hours and then the resulting NIV pellet was resuspended 0.5 mg/ml cisplatin to the original volume) or formulation C (hydrated with 6 mg/ml cisplatin, NIV were pelleted by centrifugation at 60,000 rpm for 2 hours and then the resulting NIV pellet was resuspended in saline to the original volume). Seven days later parasite burdens in the spleen, liver and bone marrow were determined.
^{a}p < 0.05 compared to control,
^{b}p < 0.01 compared to formulation A or formulation B.

### Example 5

### Sustained Release Formulations

Further studies suggest that using drug loaded NIV without eliminating unentrapped the drug like lipsomal formulations described previously in the art decreases their in vivo efficacy—based on higher efficacy against L. donovani after intravenous injection. The unentrapped drug concentration may be important as maintaining cisplatin-NIV is cisplatin at 1 mg/ml resulted in a reduction in entrapment efficiency. In vivo studies showed that treatment with processed cisplatin-NIV resulted in a reduction in tumour growth 15 days post-treatment whereas similar treatment with cisplatin solution had no effect (Table 8). Studies have shown that it is not possible to detect cisplatin 15 minutes after dosing by conventional HPLC analysis therefore the ability of the processed cisplatin-NIV to significantly reduce tumour growth, measured as a change in lung weight, indicates that effective drug concentrations must still be present at this time point or a higher initial delivery causes a sustained cellular insult. It is possible that maintaining drug loaded NIV in a drug solution means that on treatment the formulation gives a bolus dose of free cisplatin and then extended exposure to drug released from NIV during their breakdown within cells. In other cancer models a protracted exposure to fluorouracil rather than a bolus dose has been shown to improve therapeutic outcome (Hansen et al., 1996). Comparison of the efficacy of bolus cisplatin treatment and low dose consecutive treatment with cisplatin showed that both were equally active against pulmonary metastases in sarcoma bearing mice (Takeda et al., 2003).

TABLE 8
The effect of treatment with different cisplatin formulation on tumour
growth in BALB/c female mice.

| Treatment | Lung weight |
|---|---|
| Free drug (1 mg/ml) | 0.33 ± 0.02 |
| Processed cisplatin-NIV (0.5 mg/ml) | 0.25 ± 0.02** |

Mice were injected on day 0 with 2 × 10⁵ B16 F0 cells. On day 7 mice were treated by inhalation by exposing for 6.5 mins to 0.5 ml cisplatin solution (0.5 mg/ml) or processed cisplatin-NIV (0.5 mg/ml) Formulations were nebulised directly into the Spacer for 1.5 minutes using a Buxco ® nebulisation system which delivered 0.5 ml of the appropriate formulation into the Volumatic ™ Spacer and then manintained in that environment for a further 5 minutes. Mice were sacrificed on day 23 of the expt. Controls are not present for ethical reasons, as tumours may have been very large by this time point.
**p < 0.02 compared to free cisplatin treatment.

### Example 6

### Gender Differences

Data from studies in female and male mice indicate that the B16 F0 cell line may grow quicker in male mice and that male mice are more responsive to cisplatin treatment. Thus on day 14 the lungs in male mice were heavier than those of female mice and treatment by inhalation with cisplatin at 0.5 mg/ml caused a significant reduction in lung weight whereas treatment with cisplatin at 1 mg/ml had no effect on lung weight in female mice (Table 9). In humans there is evidence to suggest that the reverse is true and that females are at more risk of developing lung cancer (Bener et al., 2008) but respond better to treatment (Singh et al., 2006). Without wishing to be bound by theory the high activity of the cisplatin-NIV formulations in male mice may indicate that it may be more effective in treating males than cisplatin treatment alone.

TABLE 9
The effect of treatment with cisplatin on tumour growth in BALB/c
female mice.

| Treatment | Lung weight |
|---|---|
| Male mice | |
| Control | 0.42 ± 0.01 |
| Free drug (0.5 mg/ml) | 0.23 ± 0.02*** |
| Female mice | |
| Control | 0.17 ± 0.02 |
| Free drug (1 mg/ml) | 0.19 ± 0.01 |

Mice were injected on day 0 with 2 × 10⁵ B16 F0 cells. On day 7 mice were treated by inhalation by exposing for 6.5 mins to 0.5 ml of saline (control) or cisplatin solution (0.5 or 1.0 mg/ml). Formulations were nebulised directly into the Spacer for 1.5 minutes using a Buxco ® nebulisation system which delivered 0.5 ml of the appropriate formulation into the Volumatic ™ Spacer and then manintained in that environment for a further 5 minutes. Mice were sacrificed on day 14 of the expt.
***p < 0.02 compared to control.

### Example 7

### Treatment with Cisplatin-NIV Delays Development of Liver Metastases

A number of studies have been completed where mice were treated on day 7 (post-tumour induction) with different cisplatin formulations by inhalation and the effect on subsequent tumour burdens, based on organ weight or direct assessment of the number and size distribution of tumours, was assessed 14 days later. The lungs, liver and spleen were cut into longitudinal slices and the kidney bisected and placed in a tissue cassette. After routine processing in paraffin wax, 5 mm sections were mounted on a glass slide, dewaxed in 100% xylene and serially rehydrated in graded ethanol to water. Sections were then stained with haematoxylin and eosin and examined by light microscopy. For each organ the number and size of tiny (unmeasurable), small, medium and large tumours was determined counted in one H&E section. The size of the largest tumour deposit was also noted. The dimensions used to characterise tumours was small (0.5 to 1.4 mm), medium (1.5 to 2.9 mm), or large (>3 mm largest dimension). The percentage of tiny tumours was calculated using the total number of tumours present in the section.

In all three experiments carried out (Tables 10-12) treatment with cisplatin-NIV was associated with a lower liver weight compared to controls and mice treated with cisplatin solution. However, the results were only significantly different from control values in one of the experiments (Table 10). The weight of the lungs from mice treated with cisplatin-NIV was also consistently lower in two of the experiments (Tables 11 and 12) but there was little difference in the number and size distribution of tumours in the lungs of all three groups of mice (Tables 11 and 12). A similar number of tumours were present in the liver of control and drug treated mice but tumours were generally smaller with fewer large tumours observed in the liver of mice given cisplatin-NIV. However, these results were not significantly different from control values. Treatment with a lower dose of cisplatin-NIV (0.1 mg cisplatin/ml) was not associated with a reduction in the mean size of the largest tumour mass in the liver compared to controls or mice treated with cisplatin solution (Table 11), but treatment with the higher dose of cisplatin-NIV (0.5 mg cisplatin/ml) did result in a lower mean for both mouse strains (Table 12). These data indicate that treatment with cisplatin-NIV is more effective than free cisplatin treatment in retarding tumour development in the liver. This could be a consequence of this formulation being capable of delivering more cisplatin to the liver and thus directly inhibiting growth of tumours at this site, or that treatment with cisplatin-NIV delayed the ability of the tumour cells to metastasize from the lungs to the liver after treatment. These data indicate that treatment with cisplatin-NIV compared to cisplatin solution has a competitive advantage on disease outcome.

TABLE 10
Effect of treatment with different cisplatin formulations on tumour
development in BALB/c male mice

| | Mean weight (g ± SE) | | |
|---|---|---|---|
| Organ | Control | Cisplatin solution | Cisplatin-NIV |
| Lung | 0.25 ± 0.01 | 0.20 ± 0.01 | 0.21 ± 0.01 |
| | (100%) | (100%) | (100%) |
| Liver | 1.61 ± 0.11 | 1.53 ± 0.03 | 1.30 ± 0.04* |
| | (80%) | (100%) | (83%) |

Male BALB/c were inoculated with 1 × 10⁵ B16-F0 cells and treated by inhalation on day 7 with saline (control), cisplatin solution (1 mg cisplatin/ml) or cisplatin-NIV (0.5 mg cisplatin/ml, based on concentration of cisplatin outside the NIV). On day 21 animals were sacrificed and the weight of the liver and spleen determined. Values in parentheses show the % animals with visible metastases.

TABLE 11
Effect of treatment with different cisplatin formulations on tumour
development in BALB/c male mice.

| Organ | | Treatment | | |
|---|---|---|---|---|
| BALB/c | | | Cisplatin | |
| male | Parameter | Control | solution | Cisplatin-NIV |
| Lungs | Weight | 0.20 ± 0.01 | 0.22 ± 0.01 | 0.19 ± 0.01 |
| | Histology | Tiny 60% | Tiny 60% | Tiny 100% |
| | | S 3.6 ± 0.8 | S 2.5 ± 0.43 | S 1.83 ± 0.71 |
| | | M 0.4 ± 0.4 | M 1.0 ± 0.6 | M 0.6 ± 0.6 |
| | | L 0.6 ± 0.6 | L 0.40 ± 0.24 | L 0.4 ± 0.24 |
| | | D 0.98 ± | D 1.4 ± 0.37 × | D 1.3 ± 0.24 × |
| | | 0.22 × 0.52 ± | 1.19 ± 0.23 | 1.2 ± 0.33 |
| | | 0.18 | | |
| Liver | Weight | 1.42 ± 0.06 | 1.65 ± 0.10 | 1.29 ± 0.06 |
| | Histology | S 1.0 ± 0.31 | S 0.4 ± 0.24 | S 0.6 ± 0.45 |
| | | M 0.6 ± 0.6 | M 2.2 ± 1.56 | M 0.2 ± 0.20 |
| | | L 1.2 ± 0.58 | L 3.8 ± 2.15 | L 0.80 ± 0.58 |
| | | D 1.9 ± 0.47 × | D 2.38 ± | D 2.15 ± 0.68 × |
| | | 1.2 ± 0.58 | 1.22 × | 1.73 ± 0.53 |
| | | | 1.6 ± 0.43 | |

Male BALB/c were inoculated with 1.83 × 10⁵ B16-F0 cells and treated by inhalation on day 7 with saline (control), cisplatin solution (1 mg cisplatin/ml) or cisplatin-NIV (0.1 mg cisplatin/ml, based on cisplatin concentration outside the NIV). On day 21, animals were sacrificed and the weight of the liver and spleen determined. S, M and L refer to small medium and large in terms of tumour size, as discussed above. D refers to the dimensions of the largest tumour mass, where length × breadth is identified.

TABLE 12
Effect of treatment with different cisplatin formulations on tumour development in
BALB/c male mice.

| | | Treatment | | |
|---|---|---|---|---|
| Mouse strain | | Control | Cisplatin solution | Cisplatin-NIV |
| C57BL/6 male | Lungs | | | |
| | Weight | 0.26 ± 0.05 | 0.25 ± 0.03 | 0.20 ± 0.01 |
| | Tumours | Tiny 75% | Tiny 0% | Tiny 0% |
| | | S 1 ± 0.57 | S 1.2 ± 0.73 | S 1.33 ± 0.88 |
| | | M 0.25 ± 0.25 | M 1.0 ± 0.6 | M 0.33 ± 0.33 |
| | | L 0.25 ± 0.25 | L 0 | L 0 |
| | | D 1.80 ± 1.2 × | D 1.35 ± 0.35 × | D 1.6 ± 0.40 × |
| | | 1.65 ± 1.05 | 0.7 ± 0.20 | 1.2 ± 0.35 |
| | Liver | | | |
| | Weight | 1.86 ± 0.19 | 1.76 ± 0.16 | 1.50 ± 0.11 |
| | Tumours | S 2.2 ± 0.86 | S 0.20 ± 0.20 | S 1.67 ± 1.67 |
| | | M 4.5 ± 1.20 | M 2.8 ± 1.01 | M 1.0 ± 0.47 |
| | | L 6.0 ± 0.71 | L 6.4 ± 2.95 | L 0.67 ± 0.67 |
| | | D 4.43 ± 0.22 × | D 5.64 ± 0.88 × | D 2.70 ± 0.65 × |
| | | 3.88 ± 0.08 | 4.44 ± 0.56 | 2.63 ± 0.68 |
| BALB/c male | Lungs | | | |
| | Weight | 0.25 ± 0.03 | 0.22 ± 0.02 | 0.20 ± 0.01 |
| | Tumours | Tiny 100% | Tiny 60% | Tiny 40% |
| | | S 6.4 ± 2.11 | S 7.75 ± 1.11 | S 6.5 ± 1.3 |
| | | M 0 | M 0.5 ± 0.29 | M 0 |
| | | L 0.4 ± 0.24 | L 0 | L 0 |
| | | D 2.24 ± 0.71 × | D 1.45 ± 0.13 × | D 1.23 ± 0.09 × |
| | | 1.68 ± 0.62 | 1.08 ± 0.15 | 0.90 ± 0.19 |
| | Liver | | | |
| | Weight | 1.50 ± 0.02 | 1.74 ± 0.05 | 1.44 ± 0.11 |
| | Tumours | Tiny 0% | Tiny 0% | Tiny 25% |
| | | S 9.5 ± 2.10 | S 3.5 ± 1.32 | S 3.4 ± 3.7 |
| | | M 3.2 ± 2.27 | M 2.0 ± 1.41 | M 0.75 ± 0.75 |
| | | L 2.2 ± 0.65 | L 2.5 ± 1.50 | L 1.0 ± 1.0 |
| | | D 3.10 ± 0.09 × | D 2.63 ± 0.71 × | D 2.06 ± 0.96 × |
| | | 3.0 ± 0 | 2.3 ± 0.70 | 1.8 ± 0.70 |

Male BALB/c were inoculated with 1.0 × 10⁵ B16-F0 cells and treated by inhalation on day 7 with saline (control), cisplatin solution (1 mg cisplatin/ml) or cisplatin-NIV (0.5 mg cisplatin/ml, based on cisplatin concentration outside NIV). On day 21 animals were sacrificed and the weight of the liver and spleen and the size distribution of tumours within the tissues was determined. S, M, L and D have the same meanings as described for Table 11.

### REFERENCES

Carter, K. C., Baillie, A. J., Alexander, J. and Dolan, T. F. (1988) The therapeutic effect of sodium stibogluconate in the BALB/c mice infected with L. donovani is organ dependent. J. Pharm. Pharmacol. 40: 370-373.
Cryan S. A., Sivadas N., Garcia-Contreras L. (2007) In vivo animal models for drug delivery across the lung mucosal barrier. Adv. Drug Deliv Rev. 59: 1133-51.
Hanigan M. H., Devarajan P. (2003) Cisplatin nephrotoxicity: molecular mechanisms. Cancer Ther. 1: 47-61.
Mullen A. B., Carter K. C., Baillie A. J., (1997) Comparison of the efficacies of various formulations of amphotericin B against murine visceral leishmaniasis. Antimicrob Agents Chemother. 41: 2089-92.
Nakamura K., Yoshikawa N., Yamaguchi Y., Kagota S., Shinozuka K., Kunitomo M., (2002) Characterisation of mouse melanoma cell lines by their mortal malignancy using an experimental metastatic model. Life Sci. 70: 791-8.
Takeda K., Morioka D., Matsuo K., Endo E., Sekido J., Moroboshi T., Togo S., Shimada H. (2007) A case of successful resection after long-term medical treatment of invasive pulmonary aspergillosis following living donor liver transplantation. Transplant Proc. 39: 3505-8.
Tostmann A., Boeree M. J., Aarnoutse R. E., de Lange W. C., van der Ven A. J., Dekhuijzen R. (2008) Antituberculosis drug-induced hepatotoxicity: concise up-to-date review. J. Gastroenterol Hepatol. 23: 192-202.
Hansen R M, Ryan L, Anderson T, Krzywda B, Quebbeman E, Benson A 3rd, Haller D G, Tormey D C. (1996) Phase III study of bolus versus infusion fluorouracil with or without cisplatin in advanced colorectal cancer. J Natl Cancer Inst. 88: 668-74.
Takeda A, Tsuchiya H, Tajino T, Sato K, Kikuchi S. (2003). Inhibitory effects on pulmonary metastases of low-dose consecutive administration of cisplatin on sarcoma-bearing mice. Anticancer Res. 23: 309-13.
Bener A, Ayub H, Kakil R, Ibrahim W. (2008.) Patterns of cancer incidence among the population of qatar: a worldwide comparative study. Asian Pac J Cancer Prev. 9: 19-24.
Singh S., Parulekar W., Murray N., Feld R., Evans W. K., Tu D., Shepherd F. A. (2006) Influence of Sex on Toxicity and Treatment Outcome in Small-Cell Lung Cancer. Journal of Clinical Oncology, 23: 850-856.

Numerous references have been cited in this document. Each of these references is hereby incorporated by reference in its entirety.

This invention has been described with reference to its preferred embodiments. Variations and modifications of the invention will be obvious to those skilled in the art from the foregoing detailed description of the invention. It is intended that all of these variations and modifications be included within the scope of this invention.

1. A formulation for pulmonary delivery comprising active agent loaded non-ionic surfactant vesicles for delivery to the respiratory system.

2. A method of treating or preventing a respiratory infection or disease, such as a cancer, viral, fungal or bacterial infection, immune disease, inflammatory disease, allergy or the like comprising administering a formulation according to claim 1.

3. Use of a formulation for pulmonary drug delivery comprising active agent loaded non-ionic surfactant vesicles for delivery to the respiratory system for preventing, diagnosing or treating a respiratory condition or disease.

4. A method of treating a disease or condition of the respiratory trace, comprising administering to a region of the respiratory system, such as the lung, an effective amount of active agent loaded non-ionic surfactant vesicles.

5. The formulation according to claim 1 wherein the active agent is dispersed, dissolved or otherwise entrapped by the vesicles and preferably also dissolved with the aqueous or solvent environment surrounding the vesicles.

6. The method according to claim 4 for treating conditions such as lung cancer, asthma and chronic obstructive pulmonary disease, rhinitis or allergic rhinitis.

7. The method according to claim 6 for use in treating lung cancer.

8. The method according to claim 7 wherein the active agent is a cytotoxic agent, such as a platinum based agent.

9. The method according to claim 8 wherein the platinum based dry is selected from the group consisting of cisplatin, carboplatin, oxaliplatin, iproplatin, tetraplatin, transplatin, (cis-amminedichloro(cyclohexylamine)platinum(II)), (cis-amminedichloro(cyclohexylamine)-trans-dihydroxoplatinum(IV)), (bis-acetato-cis-amminedichloro(cyclohexylamine)platinum(IV)) and (trans-amminedichloro(cyclohexylamine)dihydroxoplatinum(IV)).
